(19) United States
(12) Patent Application Publication
(10) Pub. No.: US 2017/0112466 A1
(43) Pub. Date: Apr. 27, 2017

(54) ULTRASONIC DIAGNOSTIC APPARATUS, AND PROGRAM FOR CONTROLLING THE SAME

(71) Applicant: General Electric Company, Schenectady, NY (US)

(72) Inventors: Shunichiro Tanigawa, Tokyo (JP); Atsuko Matsunaga, Tokyo (JP); Mayumi Ito, Tokyo (JP); Arihiro Matsumoto, Tokyo (JP)

(21) Appl. No.: 15/334,617

(22) Filed: Oct. 26, 2016

(30) Foreign Application Priority Data
Oct. 27, 2015 (JP) 2015-210443

(51) Int. Cl. A61B 8/08; A61B 5/00; A61B 8/14; A61B 8/00

(52) U.S. Cl. CPC A61B 8/0858; A61B 8/54; A61B 5/4872; A61B 8/5215; A61B 8/485; A61B 8/14; A61B 8/4483; A61B 8/461

(57) ABSTRACT

To provide an ultrasonic diagnostic apparatus capable of transmitting a push pulse with conditions for transmission and a number of transmissions set suitably to a subject. An ultrasonic diagnostic apparatus comprises: an information input section 82 for inputting information on a subject to a setting section 81 based on an input at an operation device, the information relating to at least one of a condition for transmission of a push pulse and a number of transmissions of the push pulse; and a storage device storing therein correspondence information prescribing at least one of a condition for transmission of the push pulse corresponding to the information on the subject and a number of transmissions of the push pulse corresponding to the information on the subject, and the apparatus is characterized in that: the setting section 81 identifies at least one of a condition for transmission and a number of transmissions corresponding to the information on the subject input from the information input section 82 based on the correspondence information.

### FIELD OF THE INVENTION

The present invention relates to an ultrasonic diagnostic apparatus and a program for controlling the same for transmitting an ultrasonic push pulse to measure elasticity of biological tissue.

### BACKGROUND

There is known an elasticity measurement technique involving transmitting an ultrasonic pulse (push pulse) having a high sound pressure from an ultrasonic probe to biological tissue to measure the elasticity of the biological tissue (see Patent Document 1, for example). More particularly, shear waves generated in biological tissue by a push pulse are detected by an ultrasonic detecting pulse, and the velocity of propagation of the shear waves and/or the value of elasticity of the biological tissue are calculated to provide elasticity data. Then, an elasticity image having colors or the like according to the elasticity data is displayed.

### SUMMARY

The conditions for transmission and number of transmissions of a push pulse may be prefixed or selected by a user. In a subject having much subcutaneous fat or having a high fat content in a region in his/her biological tissue for which elasticity is to be measured, however, attenuation of a push pulse is high, which sometimes makes it difficult to obtain an elasticity image. In this case, the condition(s) for transmission or number of transmissions of a push pulse is modified, and the push pulse is transmitted again to attempt imaging.

For users, however, re-imaging is cumbersome, causing them to feel great stress. Therefore, it is desirable to be able to transmit a push pulse with conditions for transmission and a number of transmissions set suitably to a subject.

The invention, in one aspect, made for solving the aforementioned problem is an ultrasonic diagnostic apparatus characterized in comprising: an operation device for accepting an input from a user; a transmission control section for controlling transmission of an ultrasonic push pulse for generating shear waves to biological tissue in a subject; an information input section for inputting information on said subject to said transmission control section based on an input at said operation device, said information relating to at least one of a condition for transmission of said push pulse and a number of transmissions of said push pulse; and a storage device storing therein correspondence information prescribing at least one of a condition for transmission of said push pulse corresponding to said information on said subject and a number of transmissions of said push pulse corresponding to said information on said subject, wherein: said transmission control section identifies at least one of a condition for transmission and a number of transmissions of said push pulse corresponding to said information on said subject input from said information input section based on said correspondence information, and controls transmission of said push pulse with at least one of said identified condition for transmission and number of transmissions.

According to the invention in the aspect described above, correspondence information prescribing at least one of a condition for transmission and a number of transmissions of a push pulse corresponding to information on the subject acquired beforehand and relating to conditions for transmission of the push pulse is stored, at least one of a condition for transmission and a number of transmissions of the push pulse corresponding to information on the subject input from the information input section is identified based on the correspondence information, and transmission of the push pulse is controlled with at least one of the identified condition for transmission and number of transmissions; therefore, a push pulse may be transmitted with conditions for transmission and a number of transmissions set suitably to a subject.

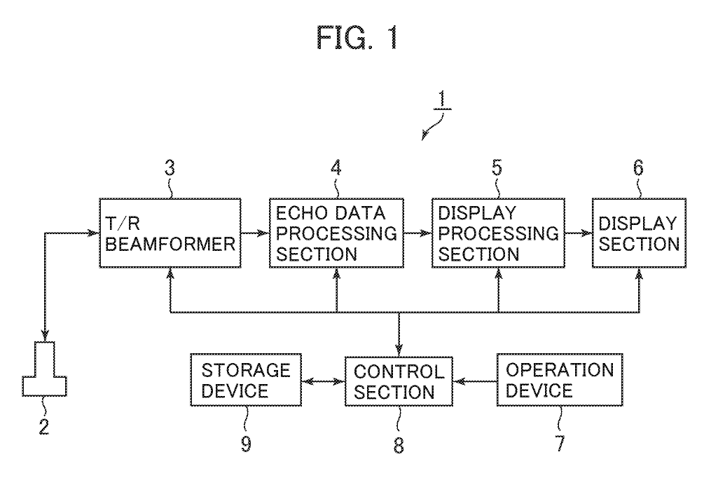

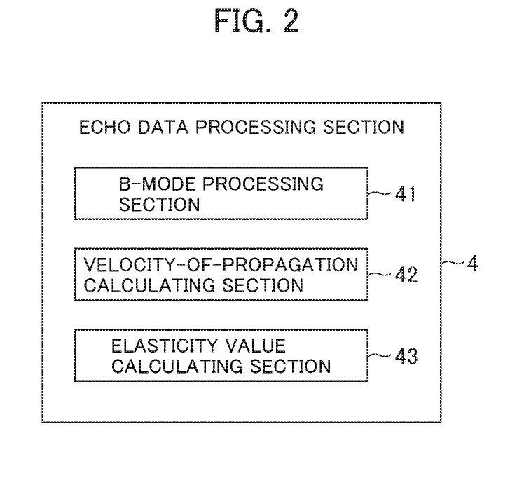

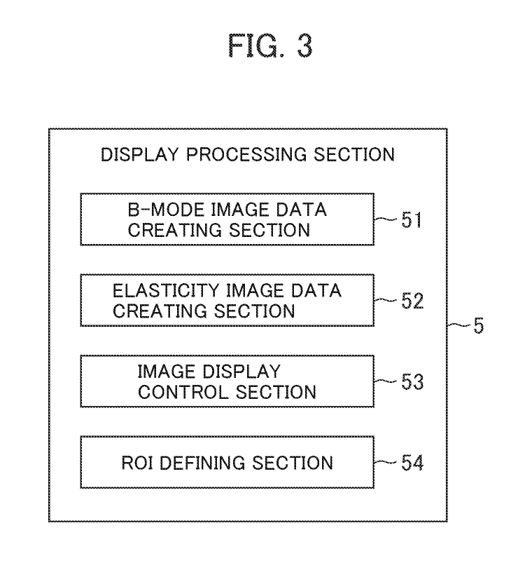

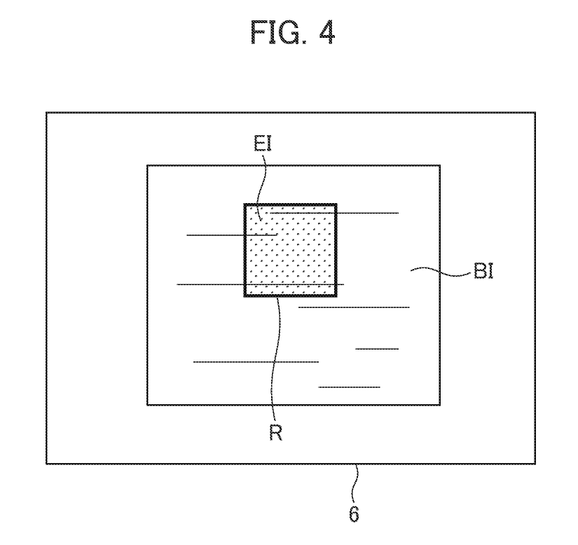

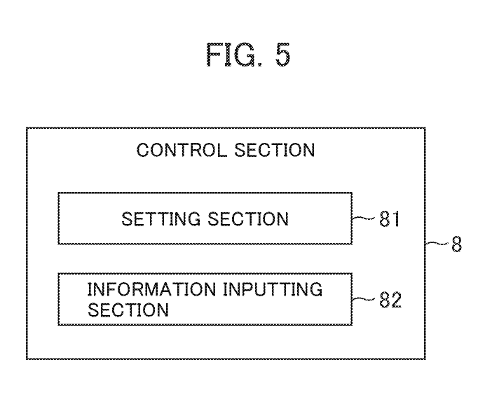

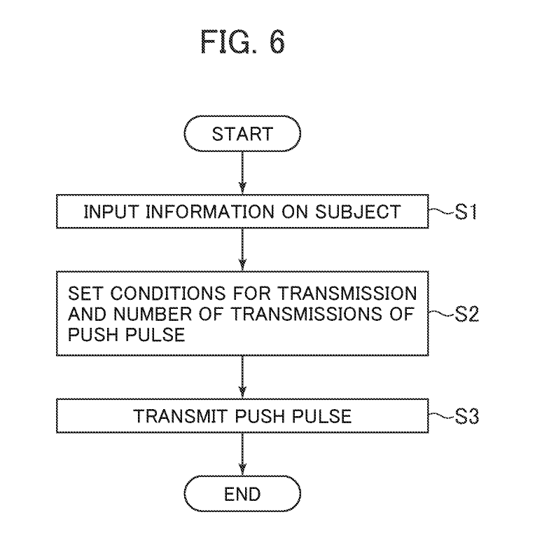

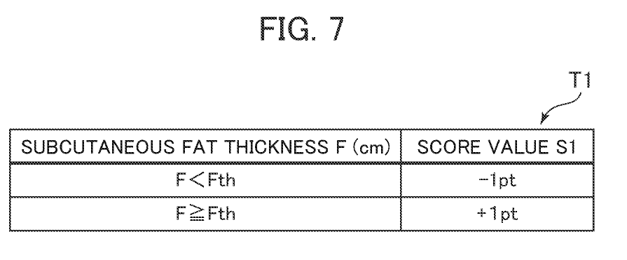

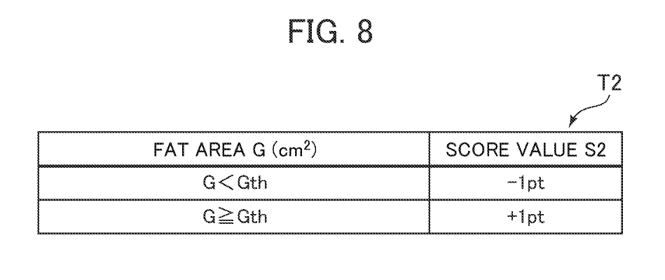

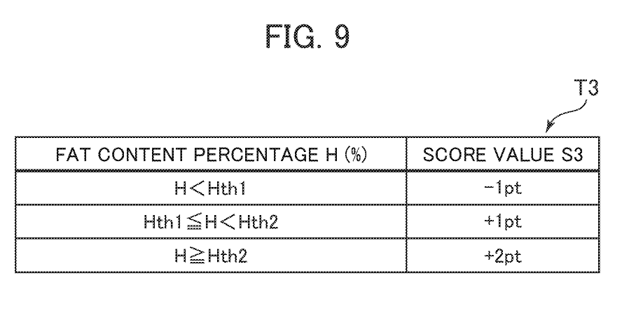

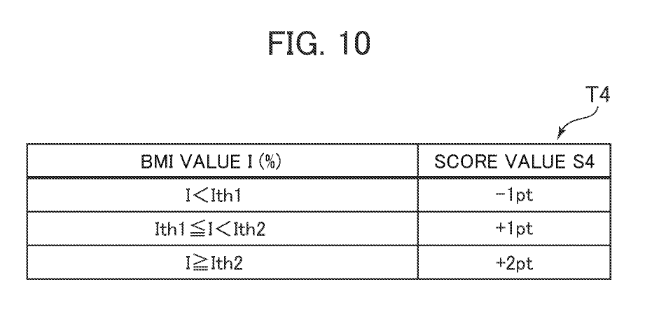

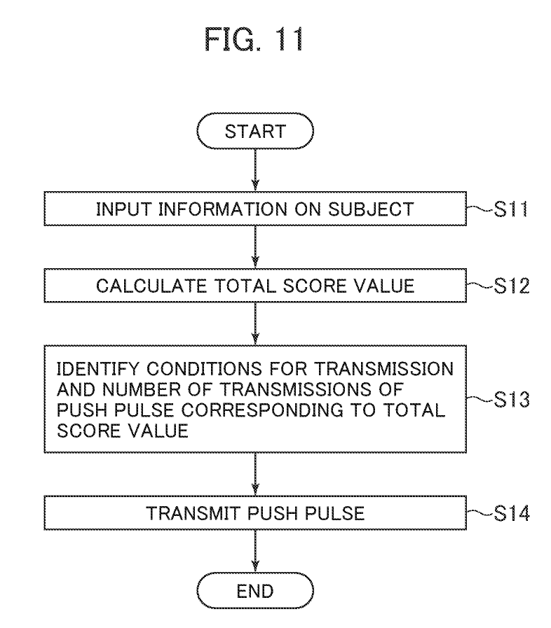

### BRIEF DESCRIPTION OF THE DRAWINGS

[FIG. 1] A block diagram showing a general configuration of an ultrasonic diagnostic apparatus, which is an exemplary embodiment of the present invention.

[FIG. 2] A block diagram showing a configuration of the echo data processing section.

[FIG. 3] A block diagram showing a configuration of the display processing section.

[FIG. 4] A diagram showing the display section in which a B-mode image and an elasticity image are displayed.

[FIG. 5] A block diagram showing part of functions of the control section.

[FIG. 6] A flow chart explaining transmission of a push pulse in a first embodiment.

[FIG. 7] A chart showing an example of a table in a second embodiment.

[FIG. 8] A chart showing another example of a table in the second embodiment.

[FIG. 9] A chart showing another example of a table in the second embodiment.

[FIG. 10] A chart showing another example of a table in the second embodiment.

[FIG. 11] A flow chart explaining transmission of a push pulse in the second embodiment.

### DETAILED DESCRIPTION

An ultrasonic diagnostic apparatus 1 shown in FIG. 1 comprises an ultrasonic probe 2, a transmission/reception (T/R) beamformer 3, an echo data processing section 4, a display processing section 5, a display section 6, an operation device 7, a control section 8, and a storage device 9. The ultrasonic diagnostic apparatus 1 has a configuration as a computer.

The ultrasonic probe 2 is an exemplary embodiment of the ultrasonic probe in the present invention, and it transmits ultrasound to biological tissue in a subject. In the ultrasonic probe 2, a plurality of ultrasonic transducers are arranged in an azimuthal direction, although not particularly shown. An ultrasonic pulse (push pulse) for generating shear waves in the biological tissue is transmitted by the ultrasonic probe 2. It is also by the ultrasonic probe 2 that an ultrasonic detecting pulse for detecting the shear waves is transmitted and echo signals thereof are received.

Moreover, it is by the ultrasonic probe 2 that an ultrasonic B-mode imaging pulse for producing a B-mode image is transmitted and echo signals thereof are received.

The T/R beamformer 3 drives the ultrasonic probe 2 based on a control signal from the control section 8 to transmit the aforementioned several kinds of ultrasonic pulses having predefined transmit parameters (transmission control function). For example, the T/R beamformer 3 transmits a push pulse according to conditions for transmission and a number of transmissions set at a setting section 81 (see FIG. 5), as will be discussed later. The T/R beamformer 3 also performs signal processing, such as phased addition processing, on echo signals of ultrasound.

The T/R beamformer 3 and setting section 81 represent an exemplary embodiment of the transmission control section in the present invention. The transmission control function is an exemplary embodiment of the transmission control function in the present invention.

The echo data processing section 4 has a B-mode processing section 41, a velocity-of-propagation calculating section 42, and an elasticity value calculating section 43, as shown in FIG. 2. The B-mode processing section 41 performs B-mode processing, such as logarithmic compression processing and envelope detection processing, on echo data output from the T/R beamformer 3 to create B-mode data.

The velocity-of-propagation calculating section 42 calculates a velocity of propagation of the shear waves based on the echo data output from the T/R beamformer 3. The velocity of propagation is calculated based on echo data obtained from a region of interest R, which will be discussed later. Therefore, a velocity of propagation of shear waves in the region of interest R is calculated. The velocity-of-propagation calculating section 42 is an exemplary embodiment of the velocity-of-propagation calculating section in the present invention.

The velocity of shear waves in biological tissue varies depending upon elasticity of the biological tissue. Therefore, a velocity of propagation depending upon elasticity of biological tissue may be obtained in the region of interest R.

The elasticity value calculating section 43 calculates an elasticity value (Young's modulus (in Pa: Pascal)) of biological tissue to which a push pulse is transmitted based on the velocity of propagation. It should be noted that only the velocity of propagation may be calculated, and the elasticity value is not necessarily calculated. Data of the velocity of propagation or data of the elasticity value will be referred to herein as elasticity data. The elasticity value calculating section 43 is an exemplary embodiment of the elasticity value calculating section in the present invention.

The display processing section 5 has a B-mode image data creating section 51, an elasticity image data creating section 52, an image display control section 53, and a region-of-interest (ROI) defining section 54, as shown in FIG. 3. The B-mode image data creating section 51 scan-converts B-mode data by a scan converter to create B-mode image data. The elasticity image data creating section 52 scan-converts elasticity data by a scan converter to create elasticity image data.

The image display control section 53 displays a B-mode image BI based on the B-mode image data in the display section 6. The image display control section 53 also displays an elasticity image EI based on the elasticity image data, as shown in FIG. 4. More particularly, the image display control section 53 combines the B-mode image data and elasticity image data to create combined image data, and displays a combined image based on the combined image data in the display section 6. The combined image is a semi-transparent color image through which the B-mode image BI in the background passes. The color image is an image having colors depending upon the velocity of propagation or elasticity value, and is the elasticity image EI having colors depending upon elasticity of biological tissue.

The region of interest R is defined by the ROI defining section 54. More particularly, the ROI defining section 54 defines the region of interest R based on an input by the user at the operation device 7. The region of interest R is a region in which shear waves are to be detected, and transmission/reception of the ultrasonic detecting pulse is performed in that region.

The display section 6 is an LCD (Liquid Crystal Display), an organic EL (Electro-Luminescence) display, or the like.

The operation device 7 is a device for accepting an input of a command and information from the user, although not particularly shown. The operation device 7 is configured to include a button, a keyboard, etc. for accepting an input of a command and information from the user, and to further include a pointing device, such as a trackball, and the like. The operation device 7 is an exemplary embodiment of the operation device in the present invention.

The control section 8 is a processor such as a CPU (Central Processing Unit). The control section 8 loads thereon programs stored in the storage device 9 to control several sections in the ultrasonic diagnostic apparatus 1. For example, the control section 8 loads thereon programs stored in the storage device 9, and executes functions of the T/R beamformer 3, echo data processing section 4, and display processing section 5 by the loaded programs.

The control section 8 may execute all of the functions of the T/R beamformer 3, all of the functions of the echo data processing section 4, and all of the functions of the display processing section 5 by the programs, or execute only part of the functions by the programs. In the case that the control section 8 executes only part of the functions, the remaining functions may be executed by hardware, such as circuitry.

The functions of the T/R beamformer 3, echo data processing section 4, and display processing section 5 may be implemented by hardware, such as circuitry.

The control section 8 also performs by the programs a setting function by the setting section 81 and an information input function by the information input section 82 shown in FIG. 5. The setting section 81 sets conditions for transmission and a number of transmissions of the push pulse. Details thereof will be discussed later. The information input section 82 inputs information on the subject relating to at least one of the condition for transmission of the push pulse and number of transmissions of the push pulse to the setting section 81. Details thereof will be discussed later. The information input section 82 is an exemplary embodiment of the information input section in the present invention.

The storage device 9 is an HDD (Hard Disk Drive), semiconductor memory, such as RAM (Random Access Memory) and ROM (Read Only Memory), and the like.

The ultrasonic diagnostic apparatus 1 may have all of the HDD, RAM and ROM as the storage device 9. The storage device 9 may also be a portable storage medium, such as a CD (Compact Disk) or a DVD (Digital Versatile Disk).

The programs executed by the control section 8 are stored in a non-transitory storage medium, such as the HDD or ROM, constituting the storage device 9. The programs may also be stored in a non-transitory storage medium having portability, such as the CD or DVD, constituting the storage device 9.

The storage device 9 stores therein correspondence information prescribing conditions for transmission of the push pulse corresponding to the information on the subject and a number of transmissions of the push pulse corresponding to the information on the subject. Details thereof will be discussed later. The storage device 9 is an exemplary embodiment of the storage device in the present invention.

Next, an operation when the ultrasonic diagnostic apparatus 1 in the present embodiment is used to perform an examination on a subject will be described. FIG. 6 is a flow chart explaining transmission of a push pulse.

First, at Step S1, the user inputs information on the subject at the operation device 7. In the present embodiment, the user inputs a height and a weight of the subject as the information on the subject. The height and weight are information acquired beforehand prior to an ultrasonic examination.

Next, at Step S2, the setting section 81 sets conditions for transmission of a push pulse and a number of transmissions of the push pulse. This will be specifically described below. The height and weight input at the operation device 7 or the like are input to the information input section 82. The information input section 82 calculates a BMI (Body Mass Index) based on the input height and weight. The BMI is information relating to body fat in the subject.

The information input section 82 inputs the BMI to the setting section 81. The BMI is information about body fat in the subject, which is information on the subject relating to the conditions for transmission of a push pulse and number of transmissions of the push pulse. The setting section 81 identifies conditions for transmission of a push pulse and a number of transmissions of the push pulse corresponding to the BMI input from the information input section 82.

The setting section 81 identifies conditions for transmission and a number of transmissions of a push pulse corresponding to the BMI input from the information input section 82 based on correspondence information prescribing conditions for transmission of a push pulse and a number of transmissions of the push pulse corresponding to a BMI. The correspondence information is a lookup table, for example, and is stored in the storage device 9.

Specifically, the conditions for transmission of a push pulse prescribed in the correspondence information and set by the setting section 81 include at least one of a transmission voltage, a central frequency, a transmit aperture, and a pulse length (pulse train length) of a push pulse. The correspondence information may prescribe all of the transmission voltage, central frequency, transmit aperture, and pulse length of a push pulse corresponding to a BMI. It should be noted that the conditions for transmission of a push pulse may include ones not listed above.

Since there is more fat as the value of BMI is greater, attenuation of a push pulse in biological tissue is higher. Accordingly, the correspondence information is set to have a higher transmission voltage, a lower central frequency, a larger transmit aperture, and a longer pulse length for a greater value of BMI. The correspondence information is set on the other hand to have a lower transmission voltage, a higher central frequency, a smaller transmit aperture, and a shorter pulse length for a smaller value of BMI.

The number of transmissions of a push pulse prescribed in the correspondence information and set by the setting section 81 may be the number (n) of frames of an elasticity image to be captured. In the case that the push pulse is transmitted m times per frame, and the elasticity image is captured in n frames, the push pulse is transmitted in m×n=x (times).

Note that when the elasticity image is obtained in a plurality of frames, the velocity of propagation and elasticity value may be measured in each frame of the elasticity image as the measured value for elasticity of biological tissue. In this case, a region for measurement is defined in each of the plurality of frames of the elasticity image, and an average value of the velocity of propagation or elasticity value in that region is calculated.

Since there is more fat in a subject as the value of BMI is greater, it is necessary to perform imaging of a larger number of frames in order to ensure that more accurate velocity of propagation and elasticity value are obtained. Accordingly, the correspondence information is set so that the number of transmissions of a push pulse (number of frames) is greater for a greater value of BMI. On the other hand, there is less fat in a subject as the value of BMI is smaller, and therefore, it is ensured that more accurate propagation velocity or elasticity value can be obtained by imaging of a smaller number of frames. Accordingly, the correspondence information is set to have a smaller number of transmissions of a push pulse (number of frames) for a smaller value of BMI.

Next, at Step S3, the T/R beamformer 3 transmits a push pulse to the subject according to the conditions for transmission and number of transmissions set by the setting section 81.

Before a push pulse is transmitted, ultrasound transmission/reception for B-mode imaging may be performed to display a B-mode image BI in the display section 6. Moreover, a region of interest R may be defined in the B-mode image BI.

The push pulse is transmitted to, for example, the outside of the region of interest R near one edge of the region of interest R in a lateral direction (X-direction).

Once a push pulse has been transmitted, ultrasonic detecting pulses for detecting shear waves generated in biological tissue by the push pulse are transmitted, and echo signals thereof are received. Then, based on the echo signals, the velocity-of-propagation calculating section 42 calculates a velocity of propagation and the elasticity image data creating section 52 creates elasticity data, so that an elasticity image EI is displayed in the region of interest R. The elasticity value calculating section 43 may calculate an elasticity value based on the velocity of propagation to display the elasticity image.

According to the present embodiment described above, the conditions for transmission and number of transmissions of a push pulse may be set suitably to body fat in a subject. More specifically, for a subject having more body fat, a push pulse may be transmitted with conditions for transmission with which attenuation of the push pulse may be suppressed as much as possible, and therefore, the user may be prevented from performing re-imaging while modifying the conditions for transmission. Moreover, since the number of transmissions of a push pulse is greater for a subject having more body fat, it is ensured that a more accurate elasticity image may be obtained. On the other hand, since the number of transmissions of a push pulse is smaller for a subject having less body fat, useless imaging may be omitted while securing an accurate elasticity image to be obtained, resulting in a shorter time taken in imaging.

Next, a variation of the first embodiment will be described. To begin with, a first variation will be described. The information on a subject input at the operation device 7 at Step S1 described above is not limited to the height and weight. For example, information identifying a subject, such as a patient ID, may be input at Step S1.

As described earlier, in the case that the information input at the operation device 7 is not information relating to body fat in a subject, the information relating to body fat in the subject is stored in the storage device 9, for example. The height and weight or BMI of the subject described earlier may be stored in the storage device 9 as the information relating to body fat in a subject.

Moreover, a subcutaneous fat thickness, a fat content percentage, a fat area, and a fat volume obtained by an MRI (Magnetic Resonance Imaging) apparatus or an X-ray CT (Computed Tomography) apparatus may be stored in the storage device 9 as the information relating to body fat in a subject.

In MRI apparatuses, information on body fat may be obtained by, for example, an MR spectroscopy technique directed to a liver. The information on body fat may also be obtained based on a fat extraction image in MRI apparatuses. According to the MR spectroscopy technique, the amount, etc. of fat may be determined with reference to water based on a spectrum of MRI signals. Moreover, a subcutaneous fat thickness, a fat content percentage, a fat area, a fat volume, etc. may be determined based on a fat image in MRI apparatuses.

Furthermore, fat stored in the liver causes X-ray absorbance to attenuate, resulting in lowering of the CT value in the liver. Therefore, the information on body fat may be calculated based on the CT value obtained in X-ray CT apparatuses.

In the case that the information relating to body fat in a subject is a subcutaneous fat thickness, a fat content percentage, a fat area, and a fat volume, correspondence information prescribing conditions for transmission of a push pulse and a number of transmissions of the push pulse corresponding thereto is stored in the storage device 9.

The information relating to body fat in a subject (information on body fat) is stored in the storage device 9 in association with information identifying the subject. Therefore, once information identifying a subject (patient identification information), such as a patient ID, has been input at the operation device 7, the information input section 82 loads information on body fat corresponding to the input patient identification information from the storage device 9. In the case that the information on body fat loaded from the storage device 9 is a height and a weight, the information input section 82 calculates a BMI based on the height and weight, and inputs the BMI to the setting section 81.

Alternatively, in the case that the information on body fat loaded from the storage device 9 is a subcutaneous fat thickness, a fat content percentage, a fat area, a fat volume, or the like, the information input section 82 inputs the information on body fat into the setting section 81.

The setting section 81 then identifies conditions for transmission of a push pulse and a number of transmissions of the push pulse corresponding to the information on body fat input from the information input section 82 based on the correspondence information in a similar manner as that described earlier.

As described earlier, the information input section 82 inputs the information relating to body fat in the subject to the setting section 81. The information relating to body fat in the subject is information on the subject relating to conditions for transmission of a push pulse and number of transmissions of the push pulse. The information input section 82 may be one for inputting the information on the subject relating to at least one of a condition for transmission of a push pulse and a number of transmissions of the push pulse to the setting section 81.

Next, a second variation will be described. As the correspondence information described above, correspondence information according to the age may be stored in the storage device 9. For example, correspondence information in a range of ages from zero to less than ‘a’ years old, correspondence information in a range of ages from ‘a’ to less than b years old, and correspondence information in a range of ages from b to less than c years old may be stored.

Here, the degree of attenuation of a push pulse in biological tissue varies among subjects having the same amount of fat but different ages. Specifically, attenuation of a push pulse in biological tissue tends to be higher for subjects of higher age. Accordingly, when correspondence information according to the age is stored, it is set to have a higher transmission voltage, a lower central frequency, a larger transmit aperture, a longer pulse length, and a greater number of transmissions of a push pulse for those having the same amount of fat but of higher age.

When correspondence information according to the age is stored, the age of the subject is input at the operation device 7 at Step S1 described above. Then, the information input section 82 inputs the age input at the operation device 7 to the setting section 81 at Step S2 described above. The setting section 81 identifies conditions for transmission and a number of transmissions of a push pulse based on correspondence information according to the age.

Next, a second embodiment will be described. It should be noted that explanation of similar matters to those in the first embodiment will be omitted.

In the ultrasonic diagnostic apparatus 1 in the present embodiment, a score value according to the amount of body fat in a subject is stored in the storage device 9 for each of a plurality of pieces of information about body fat in the subject of different kinds. Specifically, tables T1 to T4 shown in FIGS. 7 to 10 are stored.

Each of the tables T1 to T4 will now be described. The table T1 shown in FIG. 7 is a table prescribing a score value S1 depending upon the subcutaneous fat thickness F (cm). According to the table T1, when F<Fth (Fth: an arbitrary value), the score value S1 is −1 point (pt), and when F≧Fth, the score value S1 is +1 point (pt).

The table T2 shown in FIG. 8 is a table prescribing a score value S2 depending upon the fat area G (cm²). According to the table T2, when G<Gth (Gth: an arbitrary value), the score value S2 is −1 point (pt), and when G≧Gth, the score value S2 is +1 point (pt).

The table T3 shown in FIG. 9 is a table prescribing a score value S3 depending upon the fat content percentage H (%). According to the table T3, when H<Hth1 (Hth1: an arbitrary value), the score value S3 is −1 point (pt), when Hth1≦H<Hth2 (Hth2: an arbitrary value different from Hth1), the score value S3 is +1 point (pt), and when H≧Hth2, the score value S3 is +2 points (pt).

The table T4 shown in FIG. 10 is a table prescribing a score value S4 depending upon the value I of BMI. According to the table T4, when I<Ith1 (Ith1: an arbitrary value), the score value S4 is −1 point (pt), when Ith1≦H<Ith2 (Ith2: an arbitrary value different from Ith1), the score value S4 is +1 point (pt), and when I≧Ith2, the score value S4 is +2 points (pt).

In the present embodiment, correspondence information prescribing conditions for transmission of a push pulse and a number of transmissions of the push pulse corresponding to a total score value SS calculated based on the score values S1 to S4 is stored in the storage device 9. The correspondence information is a lookup table, for example. The total score value SS is information relating to body fat in a subject, and is information on the subject relating to the conditions for transmission of a push pulse and number of transmissions of the push pulse.

Transmission of a push pulse in the present embodiment will now be described based on the flow chart in FIG. 11. First, at Step S11, information identifying a subject, such as a patient ID, and the height and weight of the subject are input at the operation device 7.

Next, at Step S12, the information input section 82 calculates a total score value SS based on the information identifying the subject, and the height and weight of the subject input at Step S11. This will be specifically described below. The information input section 82 loads a subcutaneous fat thickness, a fat area, and a fat content percentage stored in the storage device 9 based on the information identifying the subject, as in the first variation of the first embodiment. The information input section 82 also calculates a BMI based on the height and weight of the subject.

The information input section 82 identifies score values S1 to S4 corresponding to the loaded subcutaneous fat thickness, fat area and fat content percentage, and calculated BMI, respectively, based on the tables T1 to T4. The information input section 82 then calculates a sum value of the score values S1 to S4 as the total score value SS.

At Step S13, the information input section 82 inputs the total score value SS to the setting section 81. The setting section 81 then identifies conditions for transmission of a push pulse and a number of transmissions of the push pulse corresponding to the total score value SS based on the correspondence information stored in the storage device 9. Next, at Step S14, the T/R beamformer 3 transmits a push pulse to the subject according to the conditions for transmission and number of transmissions set by the setting section 81.

According to the present embodiment described above, similar effects to those in the first embodiment may be achieved. Moreover, since the total score value SS is calculated based on a plurality of kinds of information regarding body fat in a subject, a push pulse may be transmitted with conditions for transmission and a number of transmissions taking account of the general state of fat deposition in the subject.

While the present invention has been described with reference to the embodiments, it will be easily recognized that the present invention may be practiced with several modifications without changing the spirit and scope thereof. For example, the information input section 82 may input information on the subject relating to at least one of a condition for transmission and a number of transmissions of a push pulse to the setting section 81. Moreover, the correspondence information may prescribe at least one of a condition for transmission of the push pulse corresponding to the information on the subject and a number of transmissions of the push pulse corresponding to the information on the subject. Furthermore, the setting section 81 may identify at least one of a condition for transmission and a number of transmissions corresponding to information on the subject input from the information input section 82, and transmission of the push pulse may be controlled with at least one of the identified condition for transmission and number of transmissions.

We claim:

1. An ultrasonic diagnostic apparatus characterized in comprising:
an operation device for accepting an input from a user;
a transmission control section for controlling transmission of an ultrasonic push pulse for generating shear waves to biological tissue in a subject;
an information input section for inputting information on said subject to said transmission control section based on an input at said operation device, said information relating to at least one of a condition for transmission of said push pulse and a number of transmissions of said push pulse; and
a storage device storing therein correspondence information prescribing at least one of a condition for transmission of said push pulse corresponding to said information on said subject and a number of transmissions of said push pulse corresponding to said information on said subject, wherein:
said transmission control section identifies at least one of a condition for transmission and a number of transmissions of said push pulse corresponding to said information on said subject input from said information input section based on said correspondence information, and controls transmission of said push pulse with at least one of said identified condition for transmission and number of transmissions.

2. The ultrasonic diagnostic apparatus as recited in claim 1, characterized in that:
said information on said subject is information relating to body fat in said subject.

3. The ultrasonic diagnostic apparatus as recited in claim 1, characterized in that:
said information on said subject is information relating to body fat in said subject and the age of said subject.

4. The ultrasonic diagnostic apparatus as recited in claim 2, characterized in that:
said information relating to body fat in said subject includes at least one of a BMI, a subcutaneous fat thickness, a fat area, a fat content percentage, and a fat volume.

5. The ultrasonic diagnostic apparatus as recited in claim 3, characterized in that:
said information relating to body fat in said subject includes at least one of a BMI, a subcutaneous fat thickness, a fat area, a fat content percentage, and a fat volume.

6. The ultrasonic diagnostic apparatus as recited in claim 1, characterized in that:
said information on said subject is information except that obtained by ultrasound transmission/reception to/from said subject.

7. The ultrasonic diagnostic apparatus as recited in claim 1, characterized in that:
conditions for transmission of said push pulse include at least one of a transmission voltage, a frequency, a transmit aperture, and a pulse length.

8. The ultrasonic diagnostic apparatus as recited in claim 1, characterized in that:
correspondence information depending upon the age of said subject is stored as said correspondence information.

9. The ultrasonic diagnostic apparatus as recited in claim 1, characterized in that:
in each of a plurality of pieces of information about body fat in a subject of different kinds, a score value depending upon an amount of body fat in said subject is set,
said information input section inputs, as said information on said subject, a total score value calculated based on said score value identified in each of said plurality of pieces of information to said transmission control section, and
said correspondence information prescribes at least one of a condition for transmission and a number of transmissions of said push pulse corresponding to said total score value calculated based on said score values.

10. The ultrasonic diagnostic apparatus as recited in claim 2, characterized in that:
in each of a plurality of pieces of information about body fat in a subject of different kinds, a score value depending upon an amount of body fat in said subject is set,
said information input section inputs, as said information on said subject, a total score value calculated based on said score value identified in each of said plurality of pieces of information to said transmission control section, and
said correspondence information prescribes at least one of a condition for transmission and a number of transmissions of said push pulse corresponding to said total score value calculated based on said score values.

11. The ultrasonic diagnostic apparatus as recited in claim 3, characterized in that:
in each of a plurality of pieces of information about body fat in a subject of different kinds, a score value depending upon an amount of body fat in said subject is set,
said information input section inputs, as said information on said subject, a total score value calculated based on said score value identified in each of said plurality of pieces of information to said transmission control section, and
said correspondence information prescribes at least one of a condition for transmission and a number of transmissions of said push pulse corresponding to said total score value calculated based on said score values.

12. The ultrasonic diagnostic apparatus as recited in claim 4, characterized in that:
in each of a plurality of pieces of information about body fat in a subject of different kinds, a score value depending upon an amount of body fat in said subject is set,
said information input section inputs, as said information on said subject, a total score value calculated based on said score value identified in each of said plurality of pieces of information to said transmission control section, and
said correspondence information prescribes at least one of a condition for transmission and a number of transmissions of said push pulse corresponding to said total score value calculated based on said score values.

13. The ultrasonic diagnostic apparatus as recited in claim 5, characterized in that:
in each of a plurality of pieces of information about body fat in a subject of different kinds, a score value depending upon an amount of body fat in said subject is set,
said information input section inputs, as said information on said subject, a total score value calculated based on said score value identified in each of said plurality of pieces of information to said transmission control section, and
said correspondence information prescribes at least one of a condition for transmission and a number of transmissions of said push pulse corresponding to said total score value calculated based on said score values.

14. The ultrasonic diagnostic apparatus as recited in claim 6, characterized in that:
in each of a plurality of pieces of information about body fat in a subject of different kinds, a score value depending upon an amount of body fat in said subject is set,
said information input section inputs, as said information on said subject, a total score value calculated based on said score value identified in each of said plurality of pieces of information to said transmission control section, and
said correspondence information prescribes at least one of a condition for transmission and a number of transmissions of said push pulse corresponding to said total score value calculated based on said score values.

15. The ultrasonic diagnostic apparatus as recited in claim 7, characterized in that:
in each of a plurality of pieces of information about body fat in a subject of different kinds, a score value depending upon an amount of body fat in said subject is set,
said information input section inputs, as said information on said subject, a total score value calculated based on said score value identified in each of said plurality of pieces of information to said transmission control section, and
said correspondence information prescribes at least one of a condition for transmission and a number of transmissions of said push pulse corresponding to said total score value calculated based on said score values.

16. The ultrasonic diagnostic apparatus as recited in claim 8, characterized in that:
in each of a plurality of pieces of information about body fat in a subject of different kinds, a score value depending upon an amount of body fat in said subject is set,
said information input section inputs, as said information on said subject, a total score value calculated based on said score value identified in each of said plurality of pieces of information to said transmission control section, and
said correspondence information prescribes at least one of a condition for transmission and a number of transmissions of said push pulse corresponding to said total score value calculated based on said score values.

17. The ultrasonic diagnostic apparatus as recited in claim 1, characterized in comprising: a velocity-of-propagation calculating section for calculating a velocity of propagation of said shear waves based on echo signals of detecting ultrasound transmitted to biological tissue in said subject for detecting said shear waves.

18. The ultrasonic diagnostic apparatus as recited in claim 2, characterized in comprising: a velocity-of-propagation calculating section for calculating a velocity of propagation of said shear waves based on echo signals of detecting ultrasound transmitted to biological tissue in said subject for detecting said shear waves.

19. The ultrasonic diagnostic apparatus as recited in claim 17, characterized in comprising: an elasticity value calculating section for calculating an elasticity value of biological tissue based on said velocity of propagation of said shear waves.

20. An ultrasonic diagnostic apparatus comprising:
an operation device for accepting an input from a user;
a storage device; and
a processor, said apparatus being characterized in that said processor executes by programs:
a transmission control function of controlling transmission of an ultrasonic push pulse for generating shear waves to biological tissue in a subject; and
an information input function of inputting information on said subject to said transmission control function based on an input at said operation device, said information relating to at least one of a condition for transmission of said push pulse and a number of transmissions of said push pulse, wherein:
in said storage device is stored correspondence information prescribing at least one of a condition for transmission of said push pulse corresponding to said information on said subject and a number of transmissions of said push pulse corresponding to said information on said subject, and
said transmission control function identifies at least one of a condition for transmission and a number of transmissions of said push pulse corresponding to said information on said subject input by said information input function based on said correspondence information, and controls transmission of said push pulse with at least one of said identified condition for transmission and number of transmissions.
